(19) United States
(12) Patent Application Publication
(10) Pub. No.: US 2026/0215728 A1
(43) Pub. Date: Jul. 30, 2026

(54) DETECTION OF CRANIOSYNOSTOSIS USING SWIR IMAGING

(71) Applicant: Children's National Medical Center, Washington, DC (US)

(72) Inventors: Richard Cha, Fulton, MD (US); Robert Keating, Washington, DC (US); Bo Ning, Rockville, MD (US); Daniel A. Donoho, Washington, DC (US)

(73) Assignee: Children's National Medical Center, Washington, DC (US)

(21) Appl. No.: 19/458,279

(22) Filed: Jan. 23, 2026

Related U.S. Application Data
(60) Provisional application No. 63/749,235, filed on Jan. 24, 2025.

(51) Int. Cl. A61B 5/00; G06T 7/00

(52) U.S. Cl. CPC A61B 5/4538; A61B 5/0062; A61B 5/0082; A61B 5/4528; G06T 7/0012; A61B 2503/045; A61B 2576/02; G06T 2207/10048; G06T 2207/20081; G06T 2207/30004

(57) ABSTRACT

A non-invasive imaging system and method for visualizing cranial sutures through overlying tissue are disclosed. The system includes a near-infrared and/or short-wave infrared illumination source, an imaging device that detects reflected short-wave infrared radiation, a linear polarizer, and a long-pass filter arranged in an optical path between a cranial region and the imaging device. The linear polarizer reduces surface-reflected light, and the long-pass filter attenuates wavelengths below a selected cutoff wavelength while passing short-wave infrared light reflected from beneath overlying tissue. Detected short-wave infrared radiation is provided to processing circuitry that generates image data representing cranial suture morphology. The disclosed approach enables evaluation of cranial sutures in the absence of ionizing radiation.

### CROSS-REFERENCE TO RELATED APPLICATIONS

The present application claims priority to U.S. Provisional Application No. 63/749,235, filed Jan. 24, 2025, which is incorporated herein by reference in its entirety.

### BACKGROUND

### Field of the Disclosure

The present disclosure relates generally to non-invasive optical imaging systems and methods. More specifically, embodiments of the present disclosure involve systems and methods that utilize near-infrared and/or short-wave infrared (SWIR) radiation to evaluate cranial sutures through overlying skin tissue.

### Description of the Related Art

Craniosynostosis is a pediatric condition characterized by the premature fusion of one or more cranial sutures, which can result in abnormal skull development. Such premature fusion may result in characteristic skull shape deformities, facial asymmetry, increased intracranial pressure, visual impairment, and cognitive deficits. Early detection of cranial suture fusion is important, as timely intervention may allow for less invasive treatment options and may mitigate long-term consequences.

Existing approaches for evaluating cranial sutures include imaging-based methods, such as computed tomography (CT) and magnetic resonance imaging (MRI). While CT imaging provides detailed anatomical information, it exposes patients to ionizing radiation, which is of particular concern for pediatric populations. Alternatively, MRI avoids ionizing radiation but is costly, time-consuming, and often requires general anesthesia for pediatric patients, limiting its practicality for routine screening and repeated evaluations.

Visual inspection of skull shape may provide indirect indications of cranial suture status. However, visual examinations do not permit direct visualization of subsurface cranial sutures and may fail to detect early-stage or subtle suture fusion. Other optical imaging techniques based on visible wavelengths are generally limited in penetration depths and may not provide sufficient contrast to visualize cranial sutures through overlying tissue.

Accordingly, there remains a need for alternative, non-invasive imaging approaches that enable quick visualization and evaluation of cranial sutures through overlying tissue without exposing pediatric patients to ionizing radiation, while providing sufficient contrast and resolution to assess suture morphology.

The foregoing Background description is provided solely for the purpose of presenting the general context of the disclosure. Any work of the inventors, to the extent described in this Background, as well as any aspects of the disclosure that may not otherwise qualify as prior art at the time of the filing, are not admitted to be prior art against the present disclosure, whether expressly or by implication.

### SUMMARY

According to an embodiment, the present disclosure relates to a non-invasive imaging system. The system includes a near-infrared and/or short-wave infrared illumination source configured to illuminate a cranial region, an imaging device configured to detect near-infrared and/or short-wave infrared (SWIR) radiation reflected from the cranial region, a linear polarizer and a long-pass filter optically coupled to the imaging device and configured to reduce surface-reflected light and enhance detection of radiation reflected from beneath overlying tissue, and processing circuitry configured to generate image data representing cranial suture morphology to non-invasively visualize cranial sutures through overlying tissue.

In one embodiment, the linear polarizer is oriented to reduce specular reflections from a surface of the overlying tissue.

In one embodiment, the long-pass filter has a cutoff wavelength in a range of approximately 900 nm to 1000 nm, in which the cutoff wavelength is selected to transmit near-infrared or short-wave infrared light above the cutoff wavelength.

In one embodiment, the linear polarizer and the long-pass filter operate together to increase image contrast of cranial suture features relative to the overlying tissue.

In one embodiment, the illumination source emits radiation within a wavelength range of approximately 700 nm to 2000 nm.

In one embodiment, the imaging device includes at least one short-wave infrared camera, a near-infrared camera, one or more photodiodes, or any combination thereof, configured to receive reflected radiation.

In one embodiment, the illumination source and the imaging device are integrated into a handheld imaging assembly.

In one embodiment, the illumination source is implemented using a ring-shaped, directional, diffuse, multi-source illumination configuration, or any combination thereof.

In one embodiment, the processing circuitry analyzes the generated image data to classify cranial suture status as patent, partially fused, or fused.

In one embodiment, the processing circuitry generates an output indicating whether additional imaging or clinical evaluation is recommended based on the analysis.

According to an embodiment, the present disclosure further relates to a method for non-invasively evaluating cranial sutures. The method includes illuminating a cranial region with near-infrared and/or short-wave infrared radiation, reducing surface-reflected light and enhancing detection of near-infrared and/or short-wave infrared light reflected from beneath overlying tissue using a linear polarizer and a long-pass filter, detecting the reflected radiation from the cranial region, and generating image data representing cranial suture morphology.

In one embodiment, reducing surface-reflected light includes orienting the linear polarizer to reduce specular reflections from a surface of the overlying tissue.

In one embodiment, enhancing detection includes filtering out wavelengths shorter than a cutoff wavelength in a range of approximately 900 nm to 1000 nm. In such embodiments, the cutoff wavelength is selected to transmit near-infrared or short-wave infrared light above the cutoff wavelength.

In one embodiment, reducing surface-reflected light and enhancing detection are performed cooperatively using the linear polarizer and the long-pass filter to increase image contrast.

In one embodiment, illuminating the cranial region includes emitting radiation within a wavelength range of approximately 700 nm to 2000 nm.

In one embodiment, detecting is performed using at least one short-wave infrared camera, a near-infrared camera, one or more photodiodes, or any combination thereof, configured to receive reflected radiation.

In one embodiment, illuminating and detecting are performed using a handheld imaging assembly.

In one embodiment, illuminating includes emitting radiation from a ring-shaped, directional, diffuse, multi-source illumination configuration, or any combination thereof.

In one embodiment, the generated image data is further processed to classify cranial suture status as patent, partially fused, or fused.

In one embodiment, the method further includes generating an output indicating whether additional imaging or clinical evaluation is recommended based on analysis of the generated image data, wherein the method is performed in the absence of ionizing radiation.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1A illustrates examples of cranial growth patterns associated with normal skull development and various types of craniosynostosis, including normocephaly, trigonocephaly, brachycephaly, anterior plagiocephaly, posterior plagiocephaly, and scaphocephaly, with arrows indicating normal growth versus arrested growth.

FIG. 1B is a block diagram illustrating an example non-invasive imaging system, including an illumination source, a skull, a linear polarizer, a long-pass filter, an imaging device, and processing circuitry.

FIG. 2A is a graph illustrating contrast as a function of tissue phantom thickness for short-wave infrared (SWIR) imaging.

FIG. 2B is a graph illustrating contrast as a function of tissue phantom thickness for visible-spectrum, red, green, blue (RGB) imaging.

FIG. 2C is an image obtained using SWIR imaging of a tissue phantom having a thickness of approximately 1.70 millimeters.

FIG. 2D is an image obtained using visible-spectrum imaging of the tissue phantom having a thickness of approximately 1.70 millimeters.

FIG. 2E is a plot profile illustrating pixel intensity as a function of distance for the SWIR image of FIG. 2C.

FIG. 2F is a plot profile illustrating pixel intensity as a function of distance for the RGB image of FIG. 2D.

FIG. 3 is a graph illustrating spatial resolution, calculated using a 1951 USAF resolution test chart, as a function of tissue phantom thickness for SWIR imaging, with an exponential curve fitted to the data.

FIG. 4A illustrates an imaging setup including an imaging device positioned above a skull model having a gap representative of a cranial suture, with a suture phantom positioned within the gap and one or more tissue phantoms placed over the skull model.

FIG. 4B is an image obtained using SWIR imaging of the skull model and suture phantom with an overlying tissue phantom having a thickness of approximately 1.59 millimeters.

FIG. 4C is an image obtained using SWIR imaging of the skull model and suture phantom with an overlying tissue phantom having a thickness of approximately 2.22 millimeters.

FIG. 4D is a plot profile illustrating pixel intensity as a function of distance for the image of FIG. 4B.

FIG. 4E is a plot profile illustrating pixel intensity as a function of distance for the image of FIG. 4C.

FIG. 5 is a block diagram illustrating an example computing device or control system that may be used to implement control circuitry for the non-invasive imaging systems and methods described herein.

### DETAILED DESCRIPTION

The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as “comprising” (i.e., open language). Reference throughout this document to “one embodiment,” “certain embodiments,” “an embodiment,” “an implementation,” “an example,” or similar terms means that a particular feature, structure, or characteristic described in connection with the embodiment is included in at least one embodiment of the present disclosure. Thus, the appearances of such phrases in various places throughout this specification are not necessarily all referring to the same embodiment. Furthermore, the particular features, structures, or characteristics may be combined in any suitable manner in one or more embodiments without limitation.

As described herein, the disclosed embodiments relate to non-invasive optical imaging systems and methods for evaluating cranial sutures using near-infrared and/or short-wave infrared (SWIR) radiation. In representative embodiments, a near-infrared and/or SWIR illumination source illuminates a cranial region and an imaging device detects reflected near-infrared and/or SWIR radiation to generate image data representing cranial suture morphology through overlying tissue, without requiring physical penetration of tissue and without exposing a subject to ionizing radiation.

As described herein, the term “short-wave infrared (SWIR) radiation” refers to electromagnetic radiation having wavelengths longer than visible light and shorter than mid-infrared radiation, including wavelengths around 940 nanometers (nm) as used in the experimental embodiments described herein. The term “cranial sutures” refers to fibrous joints between adjacent bones of the skull. The term “overlying tissue” refers to tissue positioned between an external imaging device and underlying cranial sutures, which may include the scalp and other associated soft tissue.

In some embodiments and experimental examples, visible-spectrum imaging was performed for comparison with near-infrared and/or SWIR imaging. As used herein, “visible-spectrum” or “RGB” imaging refers to imaging performed using light within the visible wavelength range, including red, green, and blue wavelength components detectable by conventional color image sensors. In such examples, RGB imaging provides a reference for evaluating contrast, penetration depth, and resolvability relative to SWIR imaging when imaging through overlying tissue.

In representative embodiments, the imaging system includes a near-infrared and/or SWIR illumination source, an imaging device configured to detect reflected near-infrared and/or SWIR radiation, and processing circuitry. The processing circuitry is configured to generate image data representing cranial suture morphology to non-invasively visualize cranial sutures through overlying tissue.

In some embodiments, the disclosed systems and methods are used to evaluate cranial sutures associated with normal and abnormal cranial growth patterns.

Although SWIR imaging has been explored for biological and medical imaging applications, many conventional approaches rely on direct reflectance imaging and are susceptible to surface-reflection artifacts that obscure subsurface anatomical features. In some cases, surface-reflected light can reduce image contrast when imaging through overlying tissue, particularly in the presence of strong surface glare. The embodiments described herein address these limitations by incorporating optical conditioning to reduce surface-reflected light and enhance detection of subsurface cranial features.

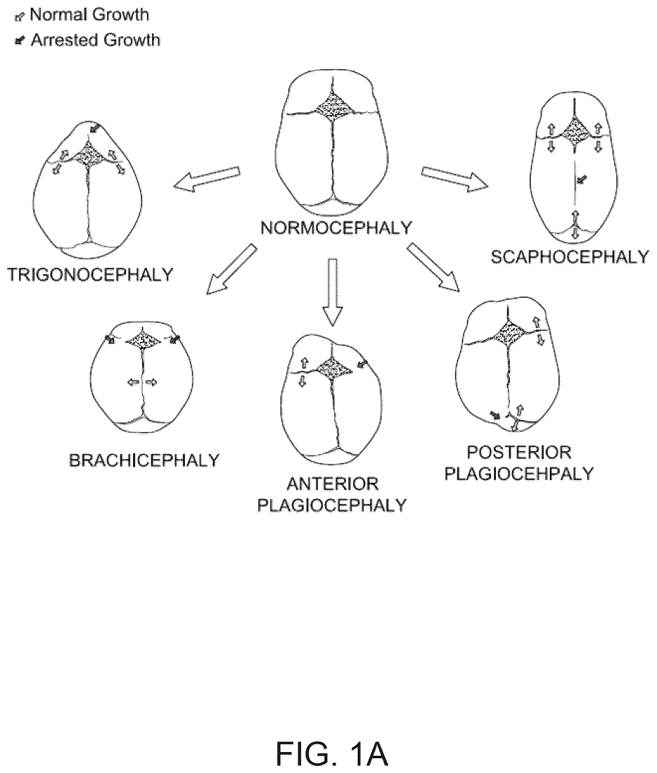

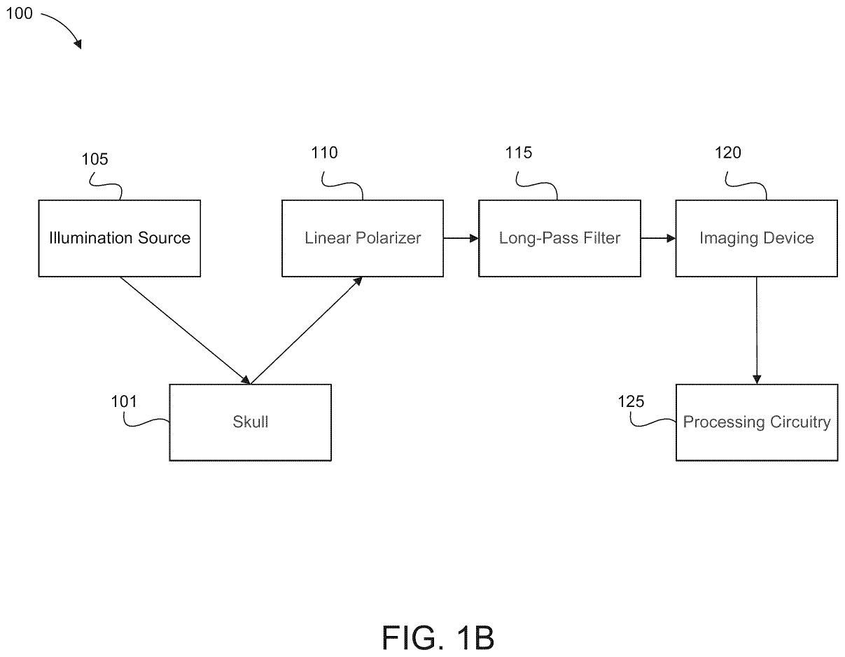

FIG. 1A illustrates representative cranial growth patterns associated with both normal skull development and craniosynostosis. As shown, normocephaly corresponds to normal cranial growth in which cranial sutures remain unfused and permit symmetric expansion of the skull. In other examples, normocephaly refers to desired skull development, in which fibrous joints (e.g., sutures) between the bones of the skull (e.g., cranium) are open and have not yet fused together.

FIG. 1A further illustrates examples of abnormal skull morphologies associated with premature cranial suture fusion, including trigonocephaly, brachycephaly, anterior plagiocephaly, posterior plagiocephaly, and scaphocephaly. Directional arrows in FIG. 1 indicate normal growth directions versus arrested growth directions resulting from premature suture fusion.

In some cases, these growth patterns illustrate the clinical importance of evaluating cranial sutures directly, as external skull shape alone may not reliably indicate underlying suture status, particularly during early stages of craniosynostosis.

In some embodiments, skin phantoms were prepared to simulate optical properties of scalp tissue. In some implementations, the skin phantoms were based on a gelatin-based matrix incorporating optical absorbers and scatterers.

In one experimental example, 10% gelatin powder (e.g., G2500, Sigma-Aldrich) and 1% agar (e.g., A1296, Sigma-Aldrich) were dissolved in 11.78 mL of deionized water and heated to 60° C. In some examples, 0.0384% India ink (e.g., Higgins Black India Ink, 44201) was added as an optical absorber, and 36.3% Intralipid- 30% (e.g., 831883443011, IL-30, Fresenius Kabi) was added as an optical scatterer. In some cases, 0.2% formaldehyde (e.g., F8775, Sigma-Aldrich) was added as an antimicrobial agent.

In some implementations, the resulting mixture was poured into a 100×15 millimeter (mm) Petri dish and placed under vacuum in a 60° C. water bath for 90 minutes, cooled to room temperature, and then cooled in a 4° C. refrigerator overnight to obtain thin skin phantom films. In some examples, a razor blade was used to cut approximately 2×1 centimeter (cm) strips of skin phantom samples. In some cases, the phantoms were stored in plastic wrap at 4° C. for up to one week prior to imaging.

In some embodiments, suture phantoms were prepared to simulate optical properties of connective tissue corresponding to cranial sutures.

In one experimental example, 1.70 g of gelatin powder was dissolved in 17.02 mL of deionized water and heated to 60° C. In some examples, 0.022 g hemoglobin (e.g., H2625, Sigma-Aldrich) and 1% Intralipid-30% were added to replicate absorption and scattering characteristics of connective tissue. In some cases, 0.2% formaldehyde was added as an antimicrobial agent.

In some implementations, the solution was poured into a 100 mm Petri dish, degassed under vacuum in a 60° C. water bath for 90 minutes, cooled to room temperature, and then cooled overnight at 4° C. In some examples, a razor blade was used to cut approximately 2 mm-wide strips of suture phantoms for imaging.

In some embodiments, the imaging device includes one or more photodiodes configured to detect near-infrared and/or SWIR radiation reflected from the illuminated cranial region. In such embodiments, the photodiodes generate electrical signals corresponding to detected reflected radiation, which may be used alone or in combination with image data generated by a SWIR camera to evaluate cranial suture morphology.

In some embodiments, SWIR imaging was performed using an Allied Vision Goldeye G-130 TEC1 SWIR camera.

In some implementations, a linear polarizer was used to reduce specular reflections, and a 950 nanometer (nm) long-pass filter was used to reject shorter wavelengths.

In some examples, a 940 nm infrared LED (e.g., Larson Electronics) was used as the illumination source. In some cases, the SWIR camera and LED were mounted on articulating arms secured to a metal table.

For comparison, in some examples, visible-spectrum imaging was performed using a Sony NEX-6 consumer-grade, mirrorless camera equipped with a 16-50 mm f/3.5-5.6 lens and a circular polarizer filter.

In some implementations, an optical enclosure constructed from ABS sheets and aluminum, 90-degree panel connectors were used to reduce ambient light during imaging. In some cases, phantom thickness was measured using an electronic caliper prior to imaging.

In some embodiments, phantom samples were placed on a 1951 USAF resolution test chart during imaging. In some examples, images were acquired with the SWIR camera at an exposure time of 6,000 microseconds, and with the RGB camera at 1/30 second using a white light source.

In some experiments, SWIR and RGB imaging performance were compared by acquiring images through skin phantoms of multiple thicknesses, including 1.36 mm, 1.59 mm, 1.70 mm, 1.85 mm, 1.95 mm, 2.22 mm, 2.23 mm, 2.68 mm, and 2.73 mm.

In some embodiments, SWIR imaging is compared with visible-spectrum imaging to evaluate contrast and penetration through overlying tissue.

FIG. 1B illustrates a block diagram of a non-invasive imaging system 100 according to some embodiments. The system 100 includes an illumination source 105 configured to emit radiation in the near-infrared and/or short-wave infrared (SWIR) wavelength range. In some embodiments, the illumination source 105 configured to emit near-infrared and/or short-wave infrared light toward a cranial region of a subject, represented by a skull 101. Reflected radiation from the skull 101 is directed along an optical path that includes a linear polarizer 110 and a long-pass filter 115, which condition the reflected light prior to detection. The conditioned reflected light is received by an imaging device 120, which generates corresponding detection signals. Processing circuitry 125 processes the detection signals to generate image data representing cranial suture morphology for non-invasive visualization through overlying tissue. In FIG. 1B, the arrows between the components illustrate an example flow of optical and signal-processing steps corresponding to the operations recited herein.

In such embodiments, the skull 101 is shown as a representative anatomical block corresponding to a cranial region of a subject and does not limit the system to any particular subject age or anatomical configuration.

In some embodiments, the imaging system 100 is used as a screening tool to assess cranial suture status of the skull 101 and to determine whether further diagnostic imaging is recommended. In such embodiments, the processing circuitry 125 may process generated image data to evaluate cranial suture morphology and to identify features indicative of suture patency, partial fusion, or complete fusion. In some embodiments, the processing circuitry 125 further classifies cranial suture status based on the identified features. In some embodiments, the processing circuitry 125 may generate an output indicating whether additional imaging or clinical evaluation is recommended based on an analysis of the generated image data. In such embodiments, the imaging, evaluation, and classification are performed without exposing the subject to ionizing radiation.

In some embodiments, the processing circuitry 125 may apply a trained model to the generated image data to classify cranial suture status. The trained model may be trained using reference image data associated with known cranial suture conditions and may output a classification result indicative of normal suture patency or suspected craniosynostosis.

In some embodiments, the generated image data is further processed by the processing circuitry 125 to enhance visualization or analysis of cranial sutures. Such processing may include one or more of contrast normalization, spatial filtering, polarization-based subtraction, or depth-dependent weighting. In some embodiments, the additional processing enhances separation between cranial suture features and surrounding bone or tissue. In some embodiments, the processed image data is used to identify, classify, or quantify cranial suture characteristics, including suture continuity, contrast, width, or degree of fusion. In some embodiments, cranial suture characteristics are compared to age-matched reference data to support screening or classification.

In some embodiments, the imaging system includes the illumination source 105 configured to illuminate a cranial region of a subject.

In some embodiments, the illumination source may emit radiation in a near-infrared or short-wave infrared wavelength range. For example, the illumination wavelength may lie within a range of approximately 700 nm to 2000 nm. Similarly, the long-pass filter may have a cutoff wavelength selected within this range to balance surface reflection reduction and subsurface penetration. Different illumination wavelengths and cutoff wavelengths may provide different tradeoffs between contrast and penetration depths.

In some embodiments, the imaging device 120 configured to detect radiation reflected from the cranial region. The imaging device 120 may comprise a SWIR camera, a near-infrared camera, one or more photodiodes, or a combination thereof. In some cases, the imaging device 120 may be configured to receive radiation reflected from the cranial region and to generate signals corresponding to the reflected radiation. In some embodiments, different detector types may be used alone or in combination to balance sensitivity, spatial resolution, and system cost. Accordingly, in some embodiments, the detected radiation is used to generate image data representing cranial suture morphology for non-invasive visualization through overlying tissue, such as skin, scalp tissue, hair, or combinations thereof.

In some embodiments, illumination of overlying tissue produces specular reflections at a tissue surface, which are mirror-like surface reflections that do not carry information about subsurface anatomical features and can obscure light reflected from beneath the tissue. In such embodiments, the linear polarizer 110 is oriented at a selected angular orientation relative to an illumination direction and an optical axis of the imaging device to attenuate specularly reflected light. In some cases, surface-reflected light is more strongly polarized than light scattered beneath the tissue. Therefore, orienting the linear polarizer 110 at the selected angular orientation may suppress surface glare while preserving signals beneath the tissue.

In some embodiments, specular reflections are reduced through a combination of optical conditioning and signal processing, including use of a linear polarizer, spectral filtering, and/or polarization-aware image processing. Reduction of specular reflections is not limited to any particular illumination geometry and may be achieved using a variety of illumination configurations.

In other implementations, reduction of specular reflections includes cross-polarization between the illumination and detection paths, wherein the linear polarizer is oriented at an angle substantially orthogonal to a polarization state of the illumination. For example, relative polarization orientations may differ by approximately 90 degrees or 270 degrees, or by approximately 0 degrees or 180 degrees, depending on the polarization reference frame.

In some embodiments, the long-pass filter 115 is positioned in the optical path at an angle or orientation selected to maintain transmission of the SWIR illumination source 105 while reducing contributions from unwanted reflected wavelengths.

In some embodiments, the long-pass filter 115 has a cutoff wavelength selected to reject reflected radiation below a short-wave infrared imaging band. For example, the cutoff wavelength may be selected in a range of approximately 900 nm to 1000 nm, such that wavelengths shorter than the cutoff are attenuated while SWIR light above the cutoff is transmitted. In some implementations, filtering out shorter-wavelength components may reduce contributions from surface scattering and ambient light, thereby improving signal quality for subsurface imaging.

In some embodiments, the long-pass filter 115 is selected to transmit light within a near-infrared and/or SWIR band extending from a possible range of approximately 1000 nm to 2500 nm.

In some embodiments, the linear polarizer 110 and the long-pass filter 115 operate together to increase image contrast of cranial suture features relative to the surrounding tissue. In some examples, the linear polarizer 110 reduces surface-reflected light originating at the tissue interface, while the long-pass filter 115 attenuates reflected wavelengths outside a selected SWIR band. This combination may reduce surface-reflected light and filtering unwanted wavelengths, enabling the system 100 to produce image data in which subsurface anatomical features on the skull 101 are more clearly distinguished. Together, these optical elements enhance detection of SWIR light reflected from beneath overlying tissue on the skull 101, thus improving visualization of cranial suture morphology.

In this manner, the arrangement of the linear polarizer 110 and the long-pass filter 115 within the optical path improves the performance of SWIR imaging for visualization of subsurface cranial features, rather than relying solely on direct SWIR reflectance.

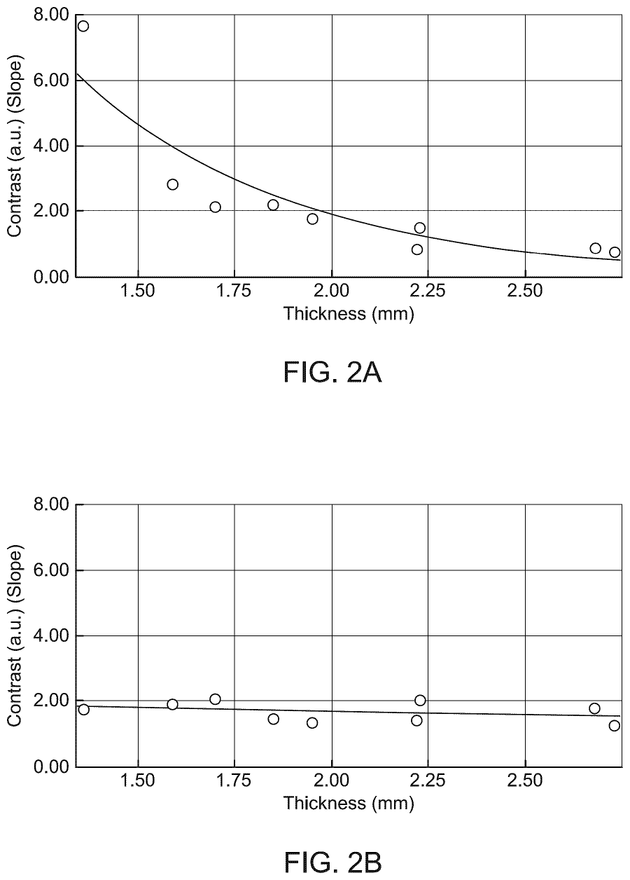

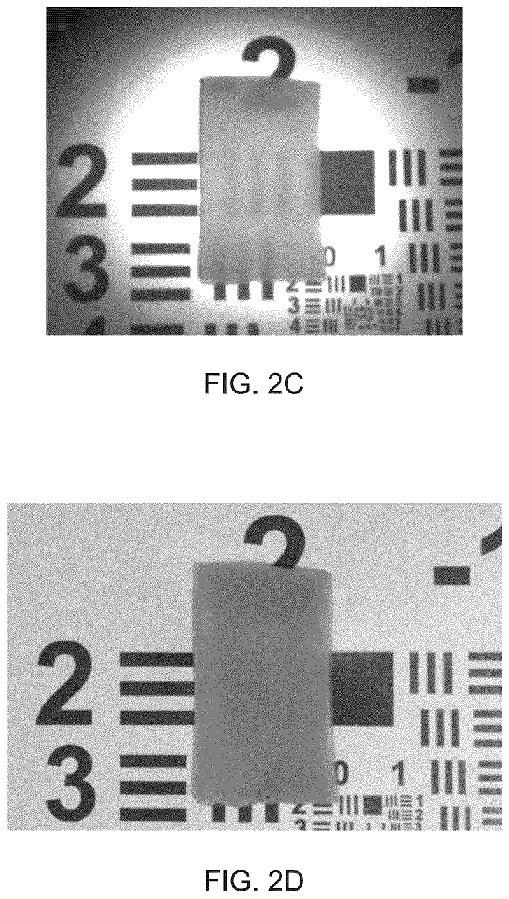

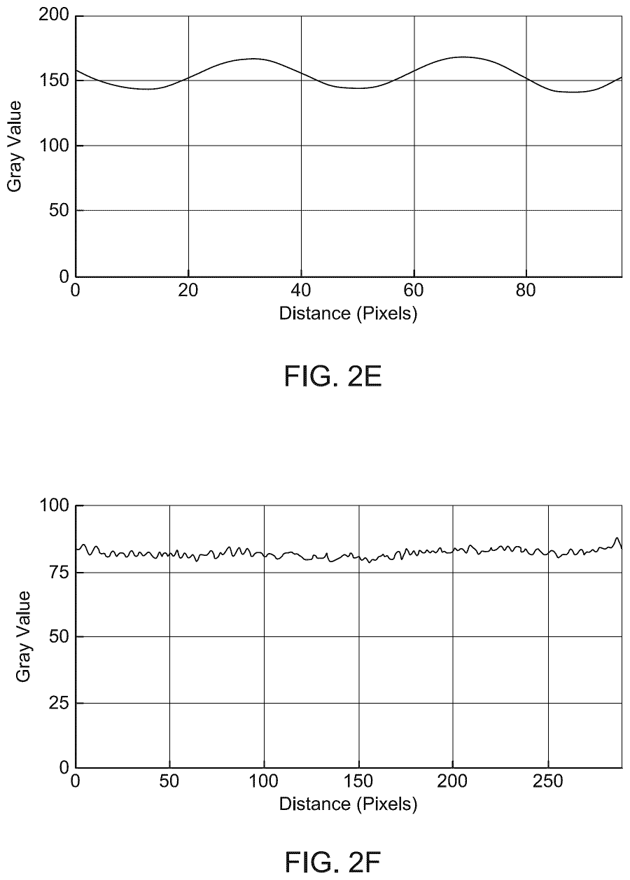

FIGS. 2A and 2B illustrate contrast as a function of tissue phantom thickness for SWIR and RGB imaging, respectively. In some implementations, contrast was calculated as an average maximum change in gray value between adjacent pixels corresponding to black and white regions, expressed in arbitrary units.

In some cases, for phantom thicknesses below approximately 2 mm, SWIR imaging exhibited greater contrast than RGB imaging. In such cases, the observed optical penetration depth for SWIR imaging was approximately 2 mm under the tested conditions.

FIGS. 2C and 2D illustrate representative images acquired using SWIR and RGB imaging, respectively, for a phantom thickness of 1.70 mm. In some examples, FIG. 2C exhibits increased brightness near a central region of the field of view due to non-uniform illumination associated with the illumination geometry of the SWIR light source, whereas FIG. 2D exhibits comparatively more uniform illumination under visible-spectrum, RGB lighting.

FIGS. 2E and 2F illustrate corresponding pixel-intensity plot profiles derived from the images of FIGS. 2C and 2D for the phantom thickness of 1.70 mm. In such embodiments, FIGS. 2E and 2F illustrate corresponding pixel-intensity plot profiles, with distance in pixels on the x-axis (approximately 0-100) and gray value on the y-axis (approximately 0-200) in arbitrary units.

In some examples, smoothing observed in the pixel-intensity plot profile of FIG. 2E was attributable to optical blurring caused by scattering within the phantom material and to non-uniform illumination intensity associated with the illumination geometry of the SWIR source, whereas the pixel-intensity plot profile of FIG. 2F exhibited reduced smoothing due to comparatively uniform visible-spectrum illumination.

In such examples, the non-uniform illumination (e.g., spotlight illumination) shown in FIG. 2C causes attenuation of sharp intensity transitions in the plot profile of FIG. 2E because the pixel-intensity profile reflects both the object contrast and the illumination profile, and a gradual change in illumination intensity across the field of view reduces the sharpness of intensity transitions in the plotted signal.

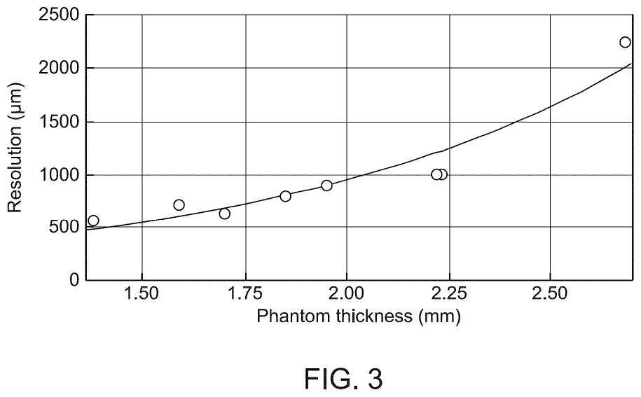

FIG. 3 illustrates a plot of spatial resolution, calculated according to a 1951 USAF resolution test chart, versus phantom thickness. In such examples, FIG. 3 plots resolution in micrometers (μm) versus phantom thickness in millimeters (mm), with an exponential curve fitted to the data.

In some embodiments, the spatial resolution was evaluated using plot profiles generated from a 1951 USAF resolution test chart. In some implementations, resolution was characterized as the smallest element for which bar patterns could be distinguished at a given phantom thickness.

In some implementations, spatial resolution may decrease with increasing phantom thickness and may drop above approximately 2.23 mm, attributable to increased scattering and limited optical penetration. In some examples, at approximately 2 mm thickness, resolvable features on the order of 1 mm were observed.

In some examples, the 2.73 mm phantom was excluded from certain resolution measurements because no resolvable bars were observed.

In some embodiments, a proof-of-concept skull phantom experiment was conducted by placing a 2 mm-wide suture phantom into a gap in a plastic skull phantom. In some implementations, skin phantoms were positioned over the skull phantom to simulate overlying tissue.

In some embodiments, a skull phantom is used to demonstrate visualization of a suture-like structure through overlying tissue.

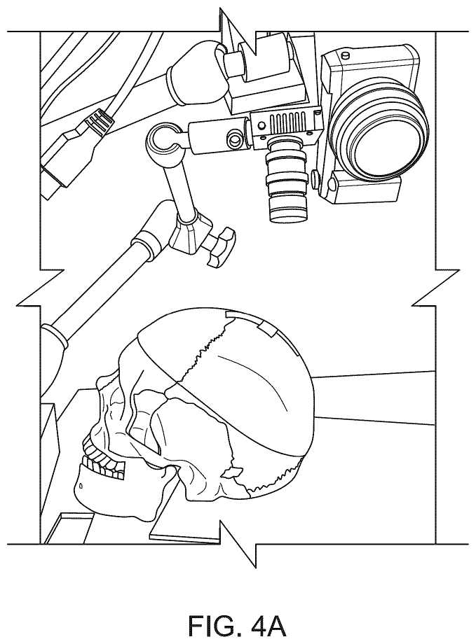

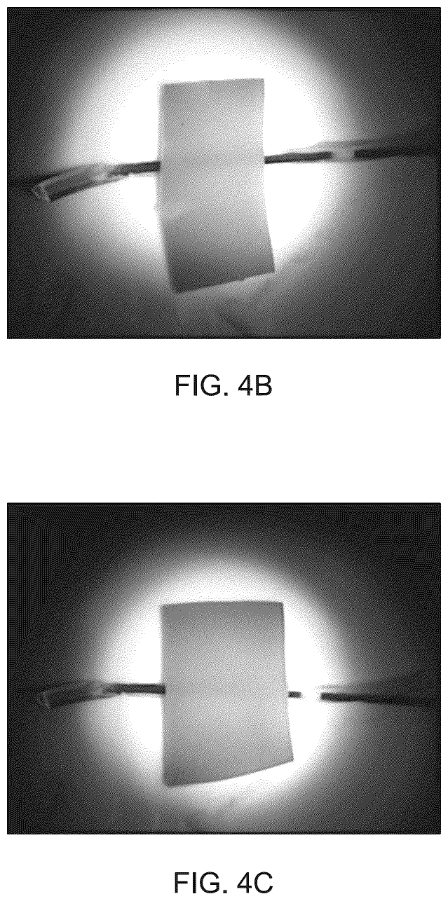

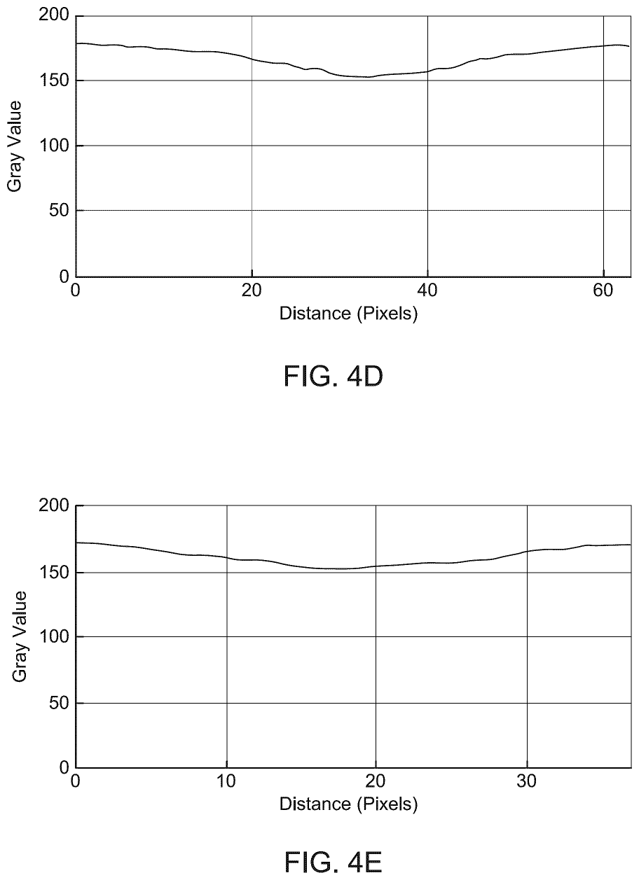

FIG. 4A illustrates an example experimental setup including a skull phantom, a suture phantom positioned within a gap of the skull phantom, a skin phantom positioned over the skull phantom, and an imaging apparatus arranged above the skull phantom.

FIGS. 4B and 4C illustrate SWIR images acquired through skin phantoms of 1.59 mm and 2.22 mm thickness, respectively. In such cases, the suture phantom was visible in both images, with reduced contrast observed at greater tissue thickness.

FIGS. 4D and 4E illustrate corresponding pixel-intensity plot profiles associated with FIGS. 4B and 4C, with distance in pixels on the x-axis (approximately 0-65) and gray value on the y-axis (approximately 0-200) in arbitrary units, demonstrating variation in signal intensity across the suture region.

Although experimental results are described with reference to phantom models, the disclosed systems and methods are applicable to imaging cranial sutures in human subjects.

In additional designs provided by the inventors, the illumination source and imaging device may be integrated into a single assembly configured for handheld operation. The handheld imaging assembly may allow an operator to position the system relative to the cranial region during use. Such configurations may be advantageous for use on a human subject by reducing alignment complexity and improving ease of positioning during imaging.

In other embodiments, the system with the illumination source and the imaging device may be implemented in non-handheld configurations, such as cart-mounted, bench-top, or fixed installations.

In some embodiments, the illumination source comprises a ring-shaped arrangement of light emitters mounted around an imaging portion of the system, such that illumination is delivered toward the cranial region from multiple angles while reflected SWIR radiation is detected by the imaging device.

In other embodiments, the illumination source is not limited to a ring-shaped configuration and may be implemented using alternative illumination arrangements, including directional, diffuse, or multi-source configurations. Such configurations may be selected to accommodate different imaging distances, fields of view, or system form factors.

In some embodiments, the illumination source includes one or more light-emitting diodes (LEDs) and one or more laser light sources, used individually or in combination as SWIR illumination sources. In some embodiments, reflected radiation is detected using a SWIR camera, a near-infrared camera, one or more photodiodes, or a combination thereof.

In this regard, these embodiments are illustrative and may be implemented with or without the experimental configurations described with respect to FIGS. 2-4.

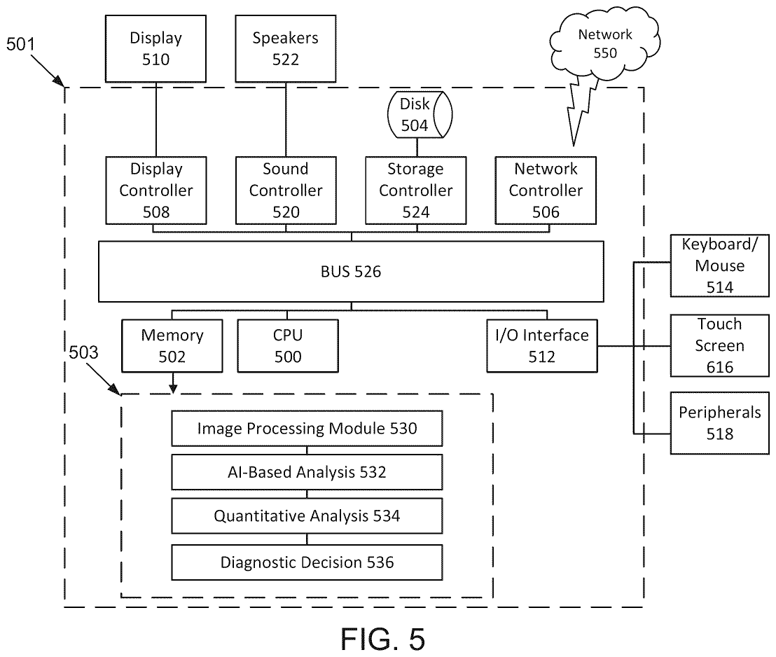

FIG. 5 illustrates an example computing device or control system that may be used to implement the processing circuitry described herein.

In FIG. 5, the device 501 includes processing circuitry, as discussed above. The processing circuitry includes one or more of the elements discussed next with reference to FIG. 5. In FIG. 5, the device 501 includes a CPU 500 which performs the processes described above/below. The process data and instructions may be stored in memory 502. These processes and instructions may also be stored on a storage medium disk 504, such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the device 501 communicates, such as a server or computer.

In some embodiments, the AI/image processing software modules 503 represent program instructions executable by CPU 500 and stored in memory 502 and/or disk 504. In the illustrated embodiment, CPU 500 may execute instructions stored in memory 502 and/or disk 504 to implement an image processing module 530 configured to perform contrast enhancement and other pre-processing operations on captured cranial image data. The CPU 500 may further implement an AI-based analysis module 532 configured to analyze the pre-processed image data to identify one or more cranial suture characteristics. In some cases, the AI-based analysis module 532 may apply one or more machine learning (ML) models to the processed image data to perform inference and extract features associated with cranial suture characteristics, including patterns indicative of suture continuity, separation, or fusion. Next, a quantitative analysis module 534 may compute one or more metrics indicative of suture patency or fusion. In some examples, the quantitative analysis module 534 may be configured to compute one or more quantitative metrics based on the extracted features, including scores or numerical measures representative of cranial suture patency, morphology, or likelihood of premature fusion. In further embodiments, a diagnostic decision module 536 may generate an assessment associated with craniosynostosis. In such implementations, the diagnostic decision module 536 may be configured to generate the craniosynostosis assessment based on the computed metrics, and to output the assessment in a human-readable and/or machine-readable form for presentation via display 510, I/O interface 512, and/or transmission over network 550.

Further, the claimed advancements may be provided as a utility application, background daemon, or component of an operating system, or combination thereof, executing in conjunction with CPU 500 and an operating system such as Microsoft Windows, UNIX, Solaris, LINUX, Apple MAC-OS and other systems known to those skilled in the art.

The hardware elements in order to achieve the device 501 may be realized by various circuitry elements, known to those skilled in the art. For example, CPU 500 may be a Xenon or Core processor from Intel of America or an Opteron processor from AMD of America, or may be other processor types that would be recognized by one of ordinary skill in the art. Alternatively, the CPU 500 may be implemented on an FPGA, ASIC, PLD or using discrete logic circuits, as one of ordinary skill in the art would recognize. Further, CPU 500 may be implemented as multiple processors cooperatively working in parallel to perform the instructions of the processes described above.

The device 501 in FIG. 5 can also include a network controller 506, such as an Intel Ethernet PRO network interface card from Intel Corporation of America, for interfacing with network 550, and to communicate with the other devices. As can be appreciated, the network 550 can be a public network, such as the Internet, or a private network such as an LAN or WAN network, or any combination thereof and can also include PSTN or ISDN sub-networks. The network 550 can also be wired, such as an Ethernet network, or can be wireless such as a cellular network including EDGE, 3G, 4G and 5G wireless cellular systems. The wireless network can also be Wi-Fi, Bluetooth, or any other wireless form of communication that is known.

The device 501 further includes a display controller 508, such as a NVIDIA GeForce GTX or Quadro graphics adaptor from NVIDIA Corporation of America for interfacing with display 510, such as an LCD monitor. A general purpose I/O interface 512 interfaces with a keyboard and/or mouse 514 as well as a touch screen panel 516 on or separate from display 510. General purpose I/O interface also connects to a variety of peripherals 518 including printers and scanners.

A sound controller 520 is also provided in the device 501 to interface with speakers/microphone 522 thereby providing sounds and/or music. In one embodiment, the device 501 can include a data acquisition (DAQ) controller to receive data corresponding to the ultrasound images.

The general purpose storage controller 524 connects the storage medium disk 504 with communication bus 526, which may be an ISA, EISA, VESA, PCI, or similar, for interconnecting all of the components of the device 501. A description of the general features and functionality of the display 510, keyboard and/or mouse 514, as well as the display controller 508, storage controller 524, network controller 506, sound controller 520, and general purpose I/O interface 512 is omitted herein for brevity as these features are known.

1. A non-invasive imaging system, comprising:
a near-infrared and/or short-wave infrared illumination source configured to illuminate a cranial region;
an imaging device configured to detect near-infrared and/or short-wave infrared radiation reflected from the cranial region;
a linear polarizer and a long-pass filter optically coupled to the imaging device and configured to reduce surface-reflected light and enhance detection of radiation reflected from beneath overlying tissue; and
processing circuitry configured to generate image data representing cranial suture morphology to non-invasively visualize cranial sutures through overlying tissue.

2. The system of claim 1, wherein the linear polarizer is oriented to reduce specular reflections from a surface of the overlying tissue.

3. The system of claim 1, wherein the long-pass filter has a cutoff wavelength in a range of approximately 900 nm to 1000 nm, the cutoff wavelength being selected to transmit near-infrared or short-wave infrared light above the cutoff wavelength.

4. The system of claim 1, wherein the linear polarizer and the long-pass filter operate together to increase image contrast of cranial suture features relative to the overlying tissue.

5. The system of claim 1, wherein the near-infrared and/or short-wave infrared illumination source emits radiation within a wavelength range of approximately 700 nm to 2000 nm.

6. The system of claim 1, wherein the imaging device comprises at least one short-wave infrared camera, a near-infrared camera, one or more photodiodes, or any combination thereof, configured to receive the reflected radiation.

7. The system of claim 1, wherein the illumination source and the imaging device are integrated into a handheld imaging assembly.

8. The system of claim 7, wherein the illumination source is implemented in a ring-shaped, directional, diffuse, multi-source illumination configuration, or any combination thereof.

9. The system of claim 1, wherein the processing circuitry processes the generated image data to classify cranial suture status as patent, partially fused, or fused.

10. The system of claim 1, wherein the processing circuitry generates an output indicating whether additional imaging or clinical evaluation is recommended based on analysis of the generated image data.

11. A method for non-invasively evaluating cranial sutures, comprising:
illuminating a cranial region with near-infrared and/or short-wave infrared radiation;
reducing surface-reflected light and enhancing detection of near-infrared and/or short-wave infrared light reflected from beneath overlying tissue using a linear polarizer and a long-pass filter;
detecting reflected near-infrared and/or short-wave infrared radiation from the cranial region using an imaging device; and
generating, using processing circuitry, image data representing cranial suture morphology based on the reflected short-wave infrared light.

12. The method of claim 11, wherein reducing surface-reflected light comprises orienting the linear polarizer to reduce specular reflections from a surface of the overlying tissue.

13. The method of claim 11, wherein enhancing detection comprises filtering out wavelengths shorter than a cutoff wavelength in a range of approximately 900 nm to 1000 nm, the cutoff wavelength being selected to transmit near-infrared or short-wave infrared light above the cutoff wavelength.

14. The method of claim 11, wherein reducing surface-reflected light and enhancing detection are performed cooperatively using the linear polarizer and the long-pass filter to increase image contrast of cranial suture features.

15. The method of claim 11, wherein illuminating comprises emitting radiation within a wavelength range of approximately 700 nm to 2000 nm.

16. The method of claim 11, wherein detecting the reflected near-infrared and/or short-wave infrared radiation is performed using at least one short-wave infrared camera, a near-infrared camera, one or more photodiodes, or any combination thereof.

17. The method of claim 11, wherein illuminating and detecting are performed using a handheld imaging assembly.

18. The method of claim 17, wherein illuminating comprises emitting radiation from a ring-shaped, directional, diffuse, multi-source illumination configuration, or any combination thereof.

19. The method of claim 11, further comprising processing the generated image data to classify cranial suture status as patent, partially fused, or fused.

20. The method of claim 11, further comprising generating an output indicating whether additional imaging or clinical evaluation is recommended, wherein the method is performed in the absence of ionizing radiation.
